(19) United States
(12) Patent Application Publication
(10) Pub. No.: US 2026/0325804 A1
(43) Pub. Date: Oct. 22, 2026

(54) SYSTEMS AND METHODS FOR IMPROVED SURGICAL DRAIN

(71) Applicant: The Brigham and Women's Hospital, Inc., Boston, MA (US)

(72) Inventors: Justin Broyles, Boston, MA (US); Goutam Gadiraju, Boston, MA (US); Matthew Prospero, Boston, MA (US)

(21) Appl. No.: 19/108,988

(22) PCT Filed: Aug. 29, 2023
(86) PCT No.: PCT/US2023/031347

§ 371 Date: Mar. 5, 2025

Related U.S. Application Data
(60) Provisional application No. 63/404,408, filed on Sep. 7, 2022.

(51) Int. Cl. A61M 27/00; A61M 35/00

(52) U.S. Cl. CPC A61M 27/00; A61M 35/10

(57) ABSTRACT

A tissue interface and method of installation are provided for connecting a drain to a patient at a skin surface. The tissue interface includes a mounting flange that includes a passage extending through the mounting flange and configured to receive at least one catheter forming a drain tube therethrough to extend through an opening in the skin surface of the patient to drain fluid from within the patient. The mouthing flange may also include a plurality of suture tabs arranged about the passage to secure the mounting flange against the skin surface of the patient and an attachment system extending about the passage and configured to removably engage the at least one catheter to secure the at least one catheter to the mounting flange.

### CROSS-REFERENCE TO RELATED APPLICATIONS

This patent application is based on, claims priority to, and incorporates herein by reference in its entirety, U.S. Provisional Patent Application Ser. No. 63/404,408, filed Sep. 7, 2022, entitled SEROSAFE DRAIN.

### STATEMENT REGARDING FEDERALLY SPONSORED RESEARCH

This invention was made with government support under FA9550-17-1-0277 and FA9550-20-1-0063 awarded by the Air Force Office of Scientific Research (AFOSR). The government has certain rights in the invention.

### BACKGROUND

Drain devices are important tools that allow for drainage of contents such as blood, serous fluid, or air from a surgical site while a patient is recovering, with an overall goal of promoting healing, wound closure, and preventing seroma formation. Typically, a drain assembly is established by placing a single, continuous catheter into the surgical site. These catheters often contain a radio-opaque strip or lining to allow for x-ray localization of the catheter after placement. Many drain devices also employ an external bulbous reservoir to collect fluids that are drained from the surgical site, often using negative pressure gradients to remove contents via suction. The collection of contents further assists surgeons in quantitative and qualitative assessment of drained fluid, allowing the care team to monitor and assess the healing process. Such drains are applied in a plurality of operations, including but not limited to abdominal, chest, breast, orthopedic, and neurosurgical procedures.

To place a drain, a small incision is made through the skin at a site near the location that is the target of the drain. This incision may be made using a scalpel to then extend a catheter through the incision. However, if the catheter that will be used in the drain features a trocar end, the catheter itself may be used to pierce the skin. In either case, a fluted end of the catheter is placed within the surgical site or cavity that is the target to be drained. The external aspect of the catheter is then pulled through the incision and secured to the skin, often by a series of surgical knots. The distal end of the external catheter may then be attached to a collection reservoir and/or negative pressure generating bulb to establish a low-pressure drainage circuit. The principal surgical site is also closed, and dressing and bandaging are completed as necessary.

Drains may be needed for periods of time ranging from days to multiple weeks following surgery. Generally, patients express substantial discomfort due to the drain. In many cases, patients indicate that the drain itself and, in particular, the catheter interfacing with tissue, at the skin or when moving internally, is much more painful than the surgical site itself.

Despite the pain, the patient must maintain the drain by fulfilling several responsibilities to ensure proper functioning and stability of the drain apparatus. For example, the patient or caregivers must ensure that the catheter is properly draining fluid and that clots do not form and occlude the catheter lumen. Notably, current drain designs promote clotting by featuring numerous narrow segments of the catheter, such as the site where the catheter exits the skin and sutures constrict the luminal diameter of the catheter. Patients or caregivers are responsible for “milking the drain tub,” an action in which the external catheter is squeezed along its length to clear contents of the drain.

Patients or caregivers must also be careful to avoid the drain getting snagged, pulled, or ripped out from the body, which is a common event. Any of these sudden movements of the drain can be very painful for the patient. If the drain apparatus is occluded or avulsed, another surgery is required to replace it. Many patients also report sensations of pain, itching, and tenderness in the skin surrounding the drain insertion site. This site may also become infected.

Therefore, there is a need for improved systems and methods for draining fluid from a patient.

### SUMMARY OF THE DISCLOSURE

The present disclosure overcomes the aforementioned drawbacks by providing systems and methods for a tissue interface that can be used with or form part of a drain device that controls against seroma formation and promotes wound healing and closure, thereby improving patient comfort, and outcomes. The tissue interface or device facilitates removal and replacement of the external catheter without the need for repeat surgery or reopening of the surgical site. A mounting flange can serve as an external skin-site attachment (ESSA) that enables secure placement of the catheter assembly without the need to occlude or narrow the catheter's luminal diameter along its length, reducing the likelihood of clot formation in the portions of the catheter external to the body. The mounting flange is designed to distribute forces and loads applied to the external tubing, reducing manipulation of the catheter assembly, thereby reducing the pain created by mechanical insults to the overall drain system. The tissue interface can include a prefabricated reservoir and elution pores to allow for the application of topical anesthetics or antibiotics to the attachment site, further reducing pain and irritation caused by the drain assembly.

In accordance with one aspect of the disclosure, a tissue interface for connecting a drain to a patient at a skin surface is provided, which can comprise a mounting flange The mounting flange can comprise a passage extending through the mounting flange. The passage can be configured to receive at least one catheter forming a drain tube therethrough to extend through an opening in the skin surface of the patient to drain fluid from within the patient. The tissue interface can also comprise a plurality of suture tabs which can be arranged about the passage to secure the mounting flange against the skin surface of the patient. The tissue interface can comprise an attachment system which can extend about the passage. The attachment system may be configured to removably engage the at least one catheter to secure the at least one catheter to the mounting flange.

In accordance with another aspect of the present disclosure, a kit is provided for connecting a drain to a patient at a skin surface. The kit includes a mounting flange comprising a passage extending through the mounting flange and configured to receive at least one catheter therethrough to extend through an opening in the skin surface of the patient to drain fluid from within the patient, a plurality of suture tabs arranged about the passage to secure the mounting flange against the skin surface of the patient, and a medical treatment system configured to deliver a medical treatment arranged therein to the skin surface proximate to the opening in the skin surface. An attachment system is configured to removably connect to the mounting flange and engage the at least one catheter to secure the at least one catheter to the mounting flange.

In yet another aspect of the disclosure, a method is provided for installing a drain in a patient. The method includes positioning at least one catheter to arrange a distal end to drain fluid from inside the patient, positioning the at least one catheter to arranged a proximal end to extend externally from the patient to dispel the fluid drained from inside the patient from the distal end from the patient, and arranging the at least one catheter to extend through a passage of a mounting flange arranged to secure the at least one catheter to a tissue or skin interface of the patient. The method further includes suturing the mounting flange to the tissue or skin interface of the patient using a plurality of suture tabs extending from the mounting flange and attaching the at least one catheter to the mounting flange using an attachment system.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### BRIEF DESCRIPTION OF THE DRAWINGS

The following drawings are provided to help illustrate various features of non-limiting examples of the disclosure, and are not intended to limit the scope of the disclosure or exclude alternative implementations.

FIG. 1 illustrates a view of a tissue interface system in accordance with the present disclosure.

FIG. 2 illustrates a view of a tissue interface connected at a skin surface in accordance with the present disclosure.

FIG. 3 illustrates a side exploded view of the tissue interface of FIG. 2 connected at the skin surface.

FIG. 4 illustrates a side exploded view of the tissue interface of FIG. 2.

FIG. 5 a mounting flange of a tissue interface in accordance with one configuration of the present disclosure.

FIG. 6 illustrates two exploded views of a tissue interface in accordance with one configuration of the present disclosure.

FIG. 7 illustrates four views of a mounting flange in accordance with one configuration of the present disclosure.

FIG. 8 illustrates four views of an attachment system design and four views of a drain coupling design in accordance with one configuration of the present disclosure.

FIG. 9 illustrates two exploded views of a tissue interface in accordance with one configuration of the present disclosure.

FIG. 10 illustrates four views of a mounting flange in accordance with one configuration of the present disclosure.

FIG. 11 illustrates four views of an attachment system design and four views of a drain coupling design in accordance with one configuration of the present disclosure.

FIG. 12 illustrates two exploded views of a tissue interface in accordance with one configuration of the present disclosure.

FIG. 13 illustrates four views of a mounting flange in accordance with one configuration of the present disclosure.

FIG. 14 illustrates two views of an attachment system design and four views of a drain coupling design in accordance with one configuration of the present disclosure.

FIG. 15 illustrates three views of a medicated patch assembly and four views of a medicated patch design in accordance with one configuration of the present disclosure.

FIG. 16 illustrates four views of a medication cap assembly and four views of a medication cap design in accordance with one configuration of the present disclosure.

### DETAILED DESCRIPTION OF THE PRESENT DISCLOSURE

Provided herein is a tissue interface which may be used to create, or be coupled with a surgical drain device used in the healthcare and biomedical devices fields, specifically in surgery, wound management, and post-operative care. The systems and methods provided herein will be described with reference to the figures, forming an instrumental yet non-limiting reduction to practice of the specification.

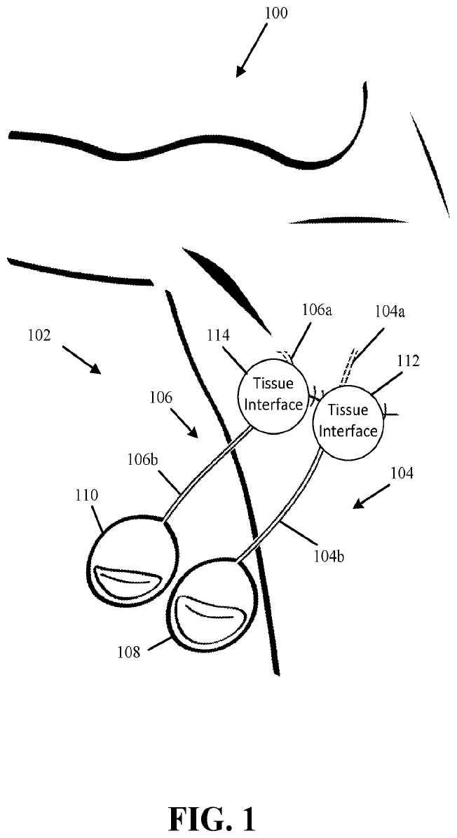

FIG. 1 is a perspective view of a patient 100 that has a drain system 102 that includes two catheters 104, 106 coupled to respective fluid-collection reservoirs 108, 110. As will be described, each catheter 104, 106 extends from an interior catheter 104a, 106a to an exterior catheter 105b, 106b as the catheter 104, 106 extends through a respective tissue interface 112, 114.

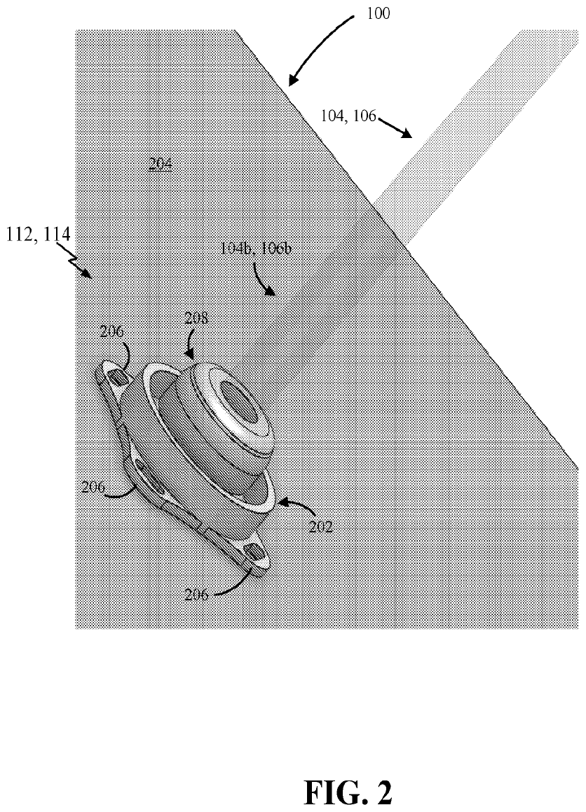

Referring now to FIG. 2, one non-limiting example of the tissue interface 112, 114 is illustrated. The tissue interface 112, 114 may include a mounting flange 202, which may be configured to be pressed against a skin surface 204 of the patient 100. The mounting flange 202 includes a plurality of suture tabs 206, which may be used to secure the mounting flange 202 to the skin surface 204 of the patient 100. As will be described, the tissue interface 112, 114 is configured to facilitate positioning of the catheter 104, 106 through the skin surface 204 of the patient while decrease the risk of avulsion compared to traditional drain configurations. The tissue interface 112, 114 may also include an attachment system 208. As will be described, the attachment system 208 may be configured to receive the external catheter 104b, 106b.

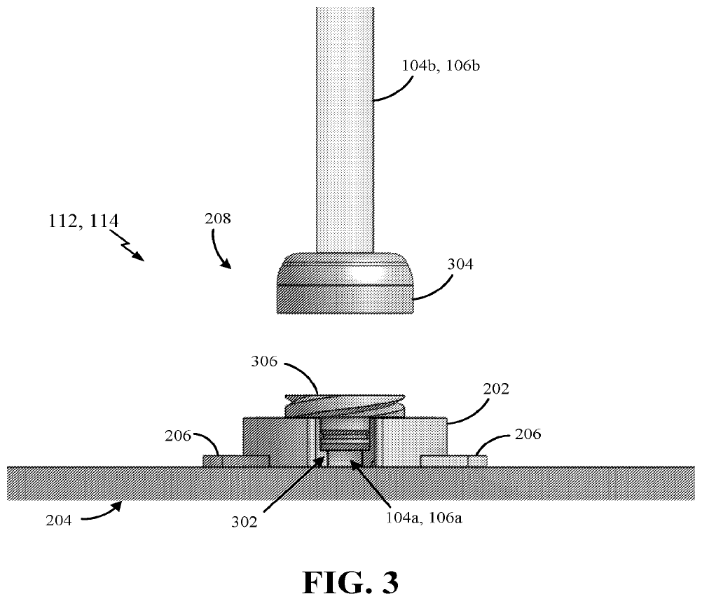

Referring to FIG. 3, a partially-disassembled view of the tissue interface 112, 114 of FIG. 2 connected at the skin surface 204. The tissue interface 112, 114 includes a passage 302. The passage 302 can extend through the mounting flange 202 and may be configured to receive the internal catheter 104a, 106a. The external catheter 104b, 106b extends through the passage 302 and can be continuous with, or coupled to, the internal catheter 104a, 106b in the passage 302. The attachment system 208 is configured to affix the catheter 104, 106 in the passage 302. The attachment system 208 may include a first lock coupling 304 configured to engage the external catheter 104b, 106b. The first lock coupling 304 is configured to then mate with a second lock coupling 306 to secure the external catheter 104b, 106b the tissue interface 112, 114. Thus, the attachment system 208 may provide a removeable connection to the external catheter 104b, 106b.

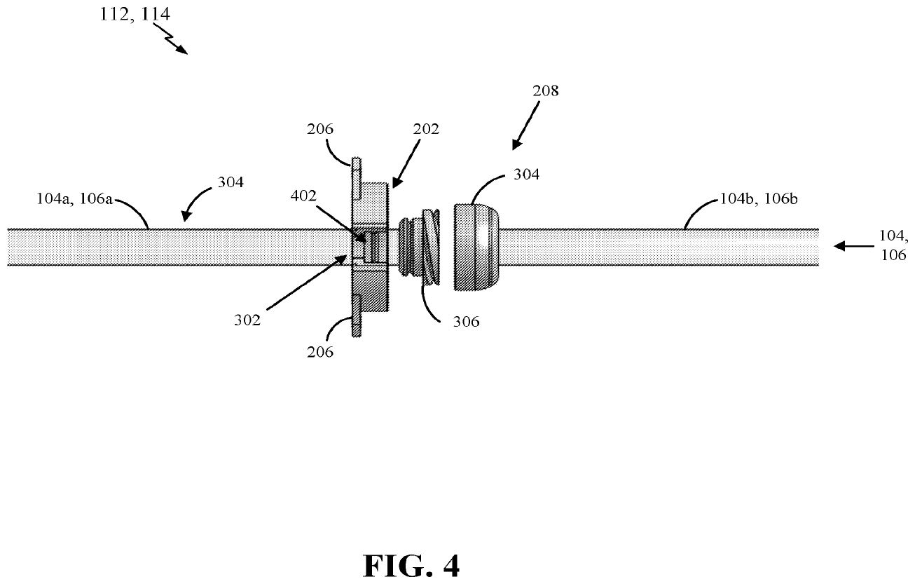

Referring now to FIG. 4, the tissue interface 112, 114 is illustrated with both the internal catheter 104a, 106a and the external catheter 104b, 106b coupled therewith. In the illustrated configuration with the second lock coupling 306 removed, a connector 402 is visible that provides a removeable connection to the mounting flange 202. The connector 402 engages the second lock coupling 306, such that the first lock coupling 304 and second lock coupling 306 can be disengaged from the mounting flange 202.

As such, a clinician can position the internal catheter 104a, 106a within the patient (100 of FIG. 1) and engage a proximal end at the mounting flange 202 to be secured via the suture tabs 206 to the skin/tissue surface. The external catheter 104b, 106b can then be extended into the first lock coupling 304 to be connected to the second lock coupling 306 to lock the external catheter 104b, 106b therebetween. The second lock coupling 306 can then be connected to the connector 402. As illustrated in FIG. 4, the connection between the second lock coupling 306 and the connector 402 can be a press or compression fitting. However, other connections, including screw, tabbed locks, or the like may also be used. As such, a system is provided that secures a distinct and separate internal catheter 104a, 106a and external catheter 104b, 106b, and securing the combined catheter 104, 106 at the skin/tissue without constricting or constraining the flow path through the tissue interface 112, 114. Alternatively, if the internal catheter 104a, 106a and external catheter 104b, 106b are one continuous lumen, the single catheter 104, 106 extends through the tissue interface 112, 114, engages the catheter 104, 106 and secures it at the skin/tissue interface, thereby reducing risk of uncontrolled movement or pulling, and reducing patient discomfort.

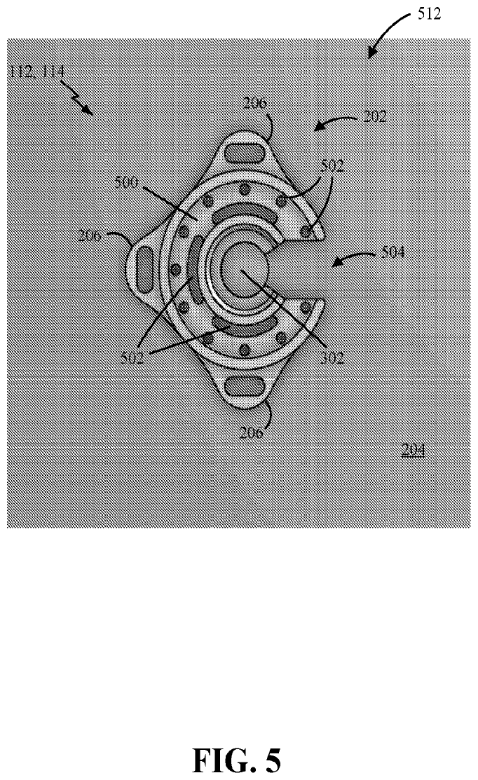

Turning now to FIG. 5, the mounting flange 202 may include a variety of optional features. For example, the plurality of suture tabs 206 arranged about the passage 302 of the mounting flange 202 may be included or removed. Also, the mounting flange 202 may include a trough 500 surrounding the passage 302, which displaces the suture tabs 206 at a more extreme periphery of the mounting flange 202 to further distribute the suture tabs 206 across the tissue.

Additionally, the mounting flange 202 may include an integrated drug dispensing or medical treatment delivery system. For example, a trough 500 may be designed to receive a drug or medical treatment. In one non-limiting example, such drug or medical treatment may comprise a topical chemical agent, which can be utilized to alleviate pains and/or prevent infection in a patient. Some non-limiting examples of topical chemical agents may include LMX 4 (4% lidocaine, Liposomal carrier), a generic form of EMLA (2.5% lidocaine and 2.5% prilocaine eutectic mixture), Topicain (4% lidocaine, amide, translucent microemulsion gel), LET (4% lidocaine, 1:2000 epinephrine, 0.5% tetracaine, gel, methylcellulose base), or BLT (20% benzocaine, 6% lidocaine, 4% tetracaine, emollient base). The trough 500 may include a plurality of perforations 502 configured to deliver the drug or medical treatment placed in the trough 500 to the skin surface 204. The perforations 502 may include any of a variety of shapes, including small perforations, such as circular-shaped openings, or large perforations, such as elongated openings. Furthermore, a combination of shapes, as illustrated, may be used in the perforations 502. For example, the elongated openings may be arranged proximate to the passage 302 to deliver an increased amount of drug or medical treatment to the opening in the skin surface relative to portions of the skin surface arranged away from the opening in the skin surface.

As also shown in FIG. 5, the mounting flange 202 may include a void 504 configured to receive the catheter 104, 106 (not shown in FIG. 5) to be positioned in the passage 302 of the mounting flange 202. In particular, if the catheter 104, 106 is continuous and not divided into physically separate internal and external catheters, the void 504 allows the catheter 104, 106 to be positioned in the patient, and then the mounting flange slid, via the void 504, around the positioned catheter 104, 106. However, as mentioned, the void 504 is optional, as the mounting flange 202 could be slid down the catheter 104, 106 along with the attachment system 208 (not shown in FIG. 5).

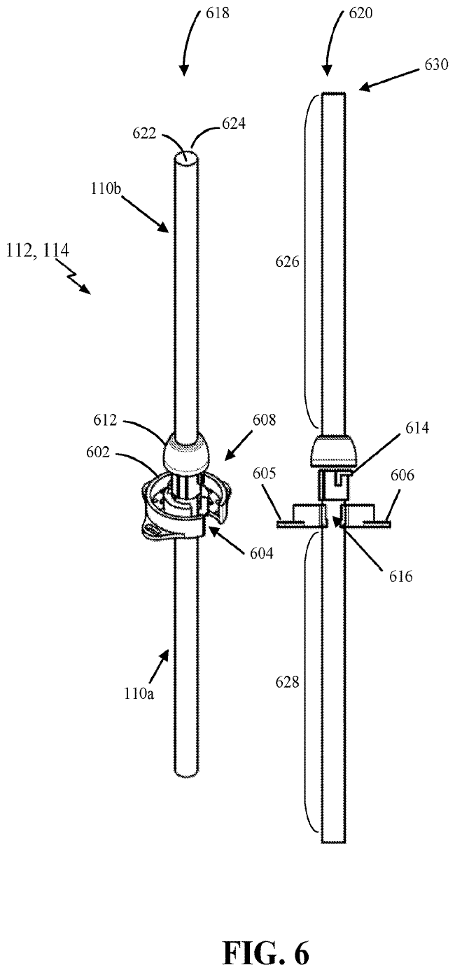

In FIG. 6, a second non-limiting example of the tissue interface 112, 114 is illustrated FIG. 6 illustrates two views of a tissue interface 112, 114 are illustrated. Shown is an exploded trimetric view 618 and an exploded side view 620 of the tissue interface 112, 114. The tissue interface 112, 114 may include a mounting flange 602, which may be configured to be pressed against a skin surface of the patient 100. The mounting flange 602 includes a plurality of suture tabs 606, The tissue interface 112, 114 may also include an attachment system 608 and an external catheter 110b. The external catheter 110b extends through a passage 616 extending through the mounting flange 602 and can be continuous with, or coupled to, an internal catheter 610a in passage 616. The attachment system 608 is configured to affix the catheter 110a, 110b in the passage 616. The attachment system 608 may include a first locking coupling 612 configured to engage the external catheter 110b. The first lock coupling 612 is configured to then mate with a second lock coupling 614 via a notch-style connection to secure the external catheter 110b to the tissue interface 112, 114. Thus, the attachment system 608 may provide a removeable connection to the external catheter 110b.

The external catheter 110b and internal catheter 110a of FIG. 6 each possess an internal luminal diameter 622, an outer diameter 624, and a length 626 628. The external catheter 110b and the internal catheter 610a possess the same internal luminal diameter 622 and external diameter 624 but may not have the same length 626, 628. A distal end 630 of the external catheter 110b may be affixed securely to a respective fluid-collection reservoir 108, 110. The proximal aspect of the second lock coupling 614 may be fluted to enable collection of fluid across a larger surface area.

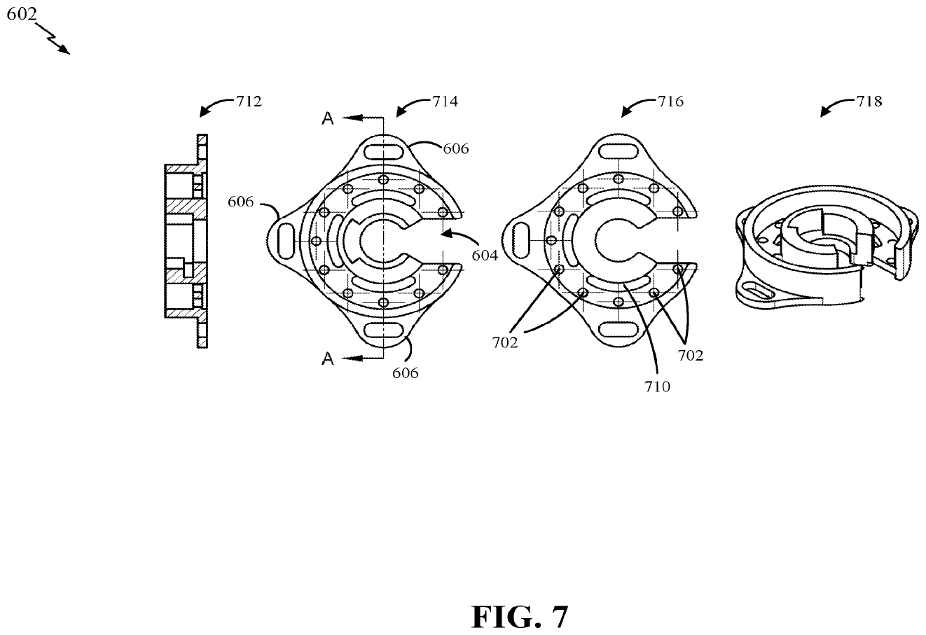

FIG. 7 illustrates four views of the mounting flange 602 of FIG. 6. Shown is a section view 712 along line A-A, a top view 714, a bottom view 716, and a trimetric view 718 of the mounting flange 602. The mounting flange 602 may include a skin-side reservoir 710 which can house a medical treatment above a skin surface of the patient 100. The mounting flange 602 may be configured to connect with the first lock coupling 612 and the second lock coupling 614 which connect via a notch-style connection

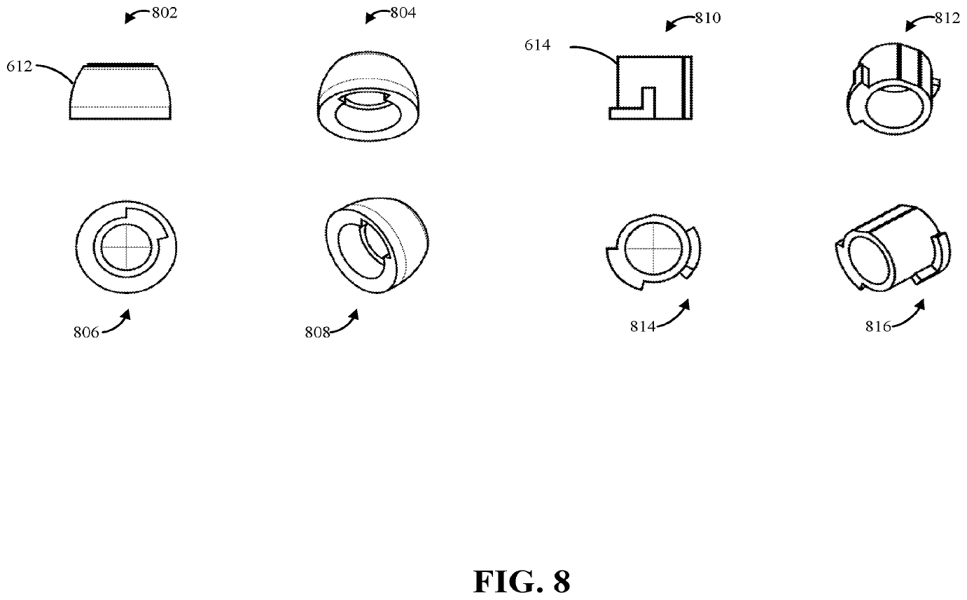

FIG. 8 illustrates four views of both the first lock coupling 612 and the second lock coupling 614 in accordance with one exemplary configuration. Shown is a side view 802, a first trimetric view 804, a bottom view 806, and a second trimetric view 808 of the first lock coupling 612, and a side view 810, a first trimetric view 812, a bottom view 814, and a second trimetric view 816 of the second lock coupling 614.

The second lock coupling 614 includes a keyed portion and the first lock coupling 612 includes a cavity. The keyed portion of the second lock coupling 614 may be configured to fit within the notch of the first lock coupling 612. Once fit within the notch of the first lock coupling 612, the second lock coupling 614 may be rotated which secures the two components to one another.

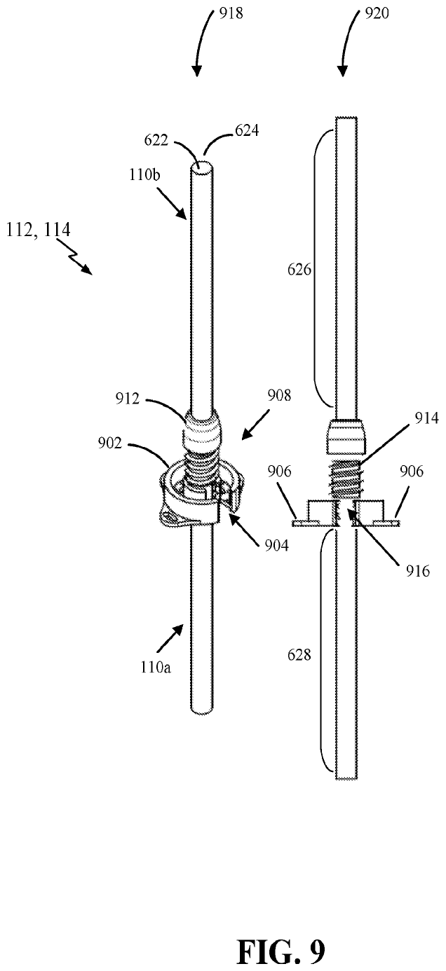

Similar to FIG. 6, FIG. 9 illustrates a third non-limiting example of the tissue interface 112, 114. Two exploded views 918, 920 of a tissue interface 112, 114 are illustrated. Instead of a notch-style connection, an attachment system 908 includes a first lock coupling 912 and a second lock coupling 914 that are configured to mate via a screw-style connection to secure an external catheter 110b to the tissue interface 112, 114. Thus, the attachment system 908 may provide a removeable connection to the external catheter 110b.

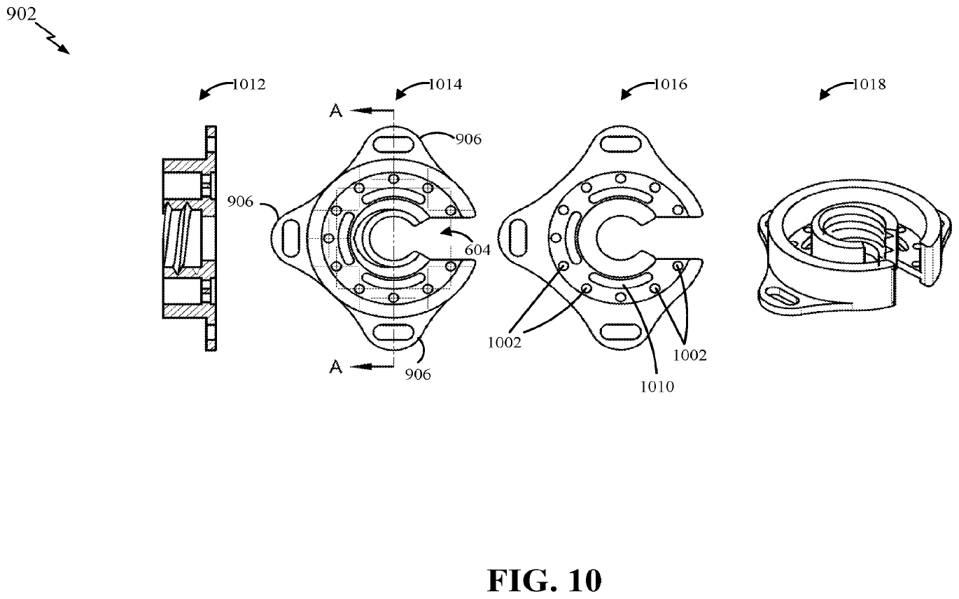

FIG. 10 illustrates four views of the mounting flange 902 of FIG. 9. Shown is a section view 1012 along line A-A, a top view 1014, a bottom view 1016, and a trimetric view 1018 of the mounting flange 902. The mounting flange 902 may include a skin-side reservoir 910 which can house a medical treatment above a skin surface of the patient 100. The mounting flange 902 may be configured to connect with the first lock coupling 912 and the second lock coupling 614 which connect via a screw-style connection.

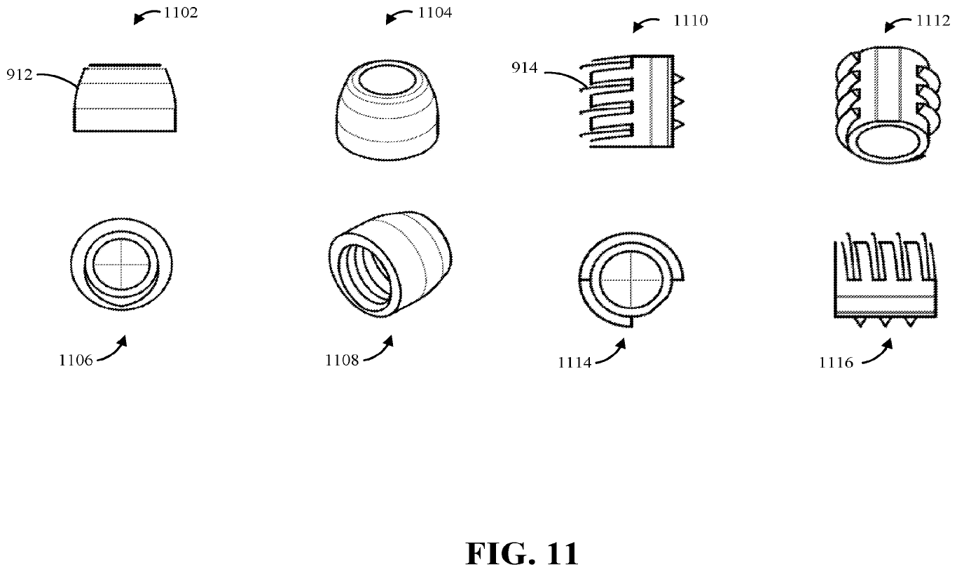

FIG. 11 illustrates four views of both the first lock coupling 912 and the second lock coupling 914 in accordance with one exemplary configuration. Shown is a side view 1102, a first trimetric view 1104, a bottom view 1106, and a second trimetric view 808 of the first lock coupling 912, and a side view 1110, a first trimetric view 1112, a bottom view 11814, and a second trimetric view 1116 of the second lock coupling 914.

The second lock coupling 914 includes external threads and the first lock coupling 912 includes internal threads. The external threads of the second lock coupling 914 may be configured to rotate within the first lock coupling 912, engaging both threads together. The second lock coupling 914 may continue to be rotated within the first lock coupling 912 until the two components are secured to one another.

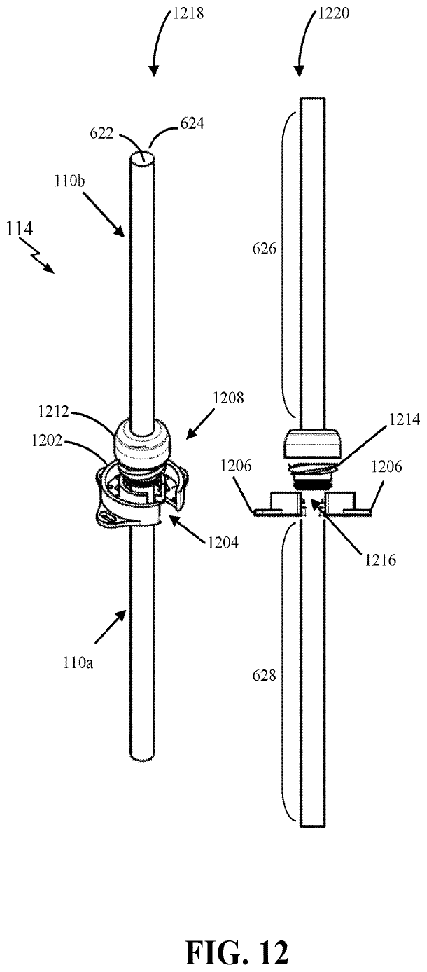

Similar to FIG. 6, FIG. 12 illustrates a fourth non-limiting example of the tissue interface 112, 114. Two exploded views 1218, 1220 of the tissue interface 112, 114 are illustrated. Instead of a notch-style connection, an attachment system 1208 includes a first lock coupling 1212 and a second lock coupling 1214 that are configured to mate via a Luer lock connection to secure an external catheter 110b to the tissue interface 112, 114. Thus, the attachment system 1208 may provide a removeable connection to the external catheter 110b.

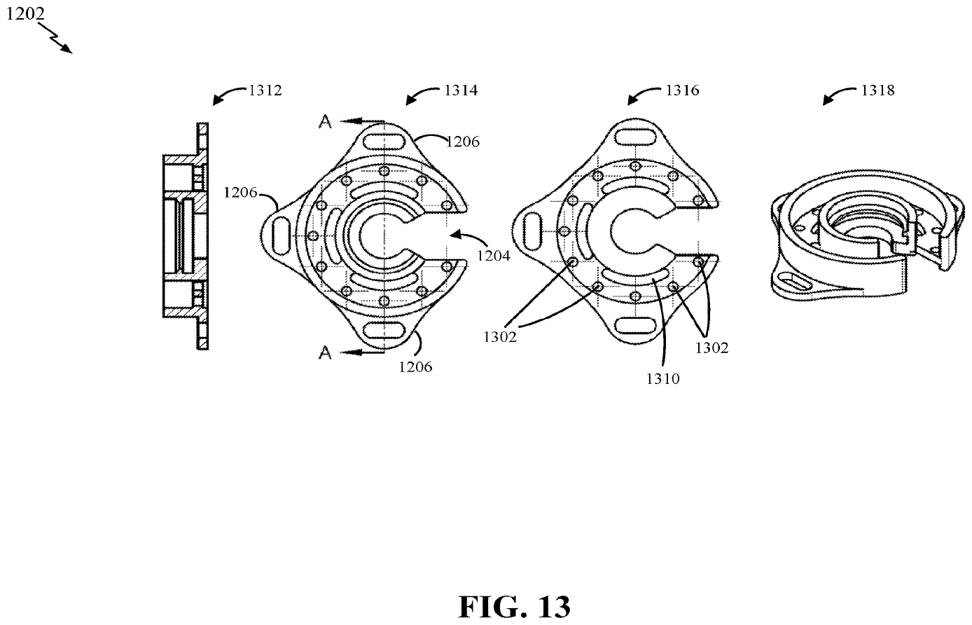

FIG. 13 illustrates four views of the mounting flange 1202 of FIG. 12. Shown is a section view 1312 along line A-A, a top view 1314, a bottom view 1316, and a trimetric view 1318 of the mounting flange 1202. The mounting flange 1202 may include a skin-side reservoir 1210 which can house a medical treatment above a skin surface of the patient 100. The mounting flange 1202 may be configured to connect with the first lock coupling 1212 and the second lock coupling 1214 which connect via a Luer lock connection.

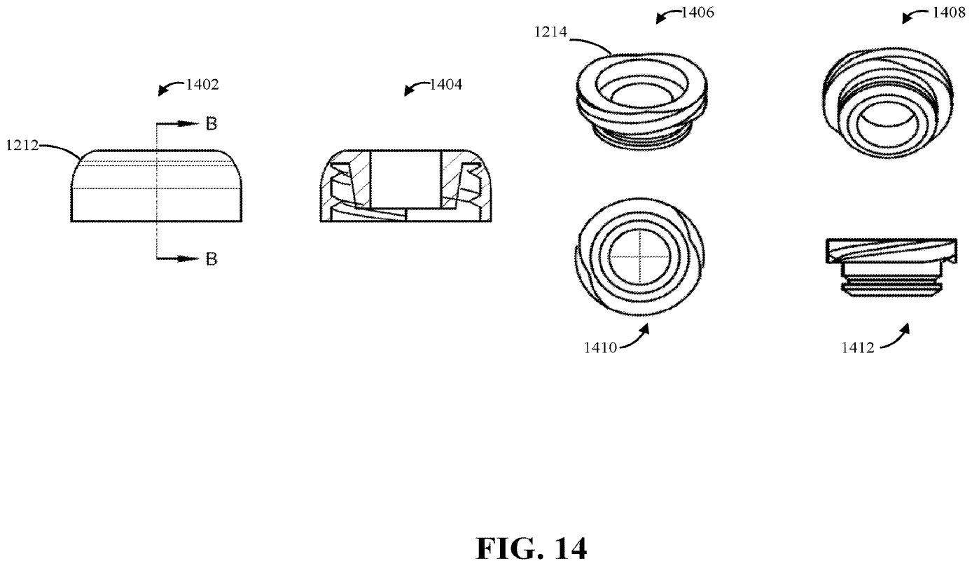

FIG. 14 illustrates two views of the first lock coupling 1212 and four views of the second lock coupling 1214 in accordance with one exemplary configuration. Shown is a side view 1402, a first trimetric view 1404, a bottom view 1406, and a second trimetric view 1408 of the first lock coupling 1212, and a side view 1410, a first trimetric view 1412, a bottom view 1414, and a second trimetric view 1416 of the second lock coupling 1214.

The second lock coupling 1214 includes external threads and an external tapered portion. The first lock coupling 1212 includes internal threads and an internal tapered portion. The external threads of the second lock coupling 1214 may be configured to rotate within the first lock coupling 1212, engaging both the internal and external threads and tapered portions together. The second lock coupling 1214 may continue to be rotated within the first lock coupling 1212 until the two components are secured to one another.

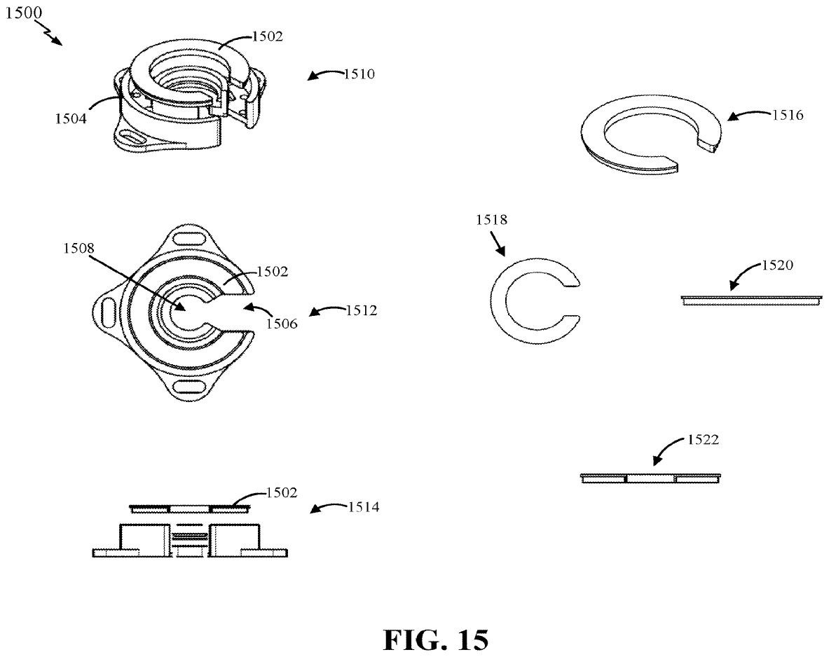

FIG. 15 illustrates three views of a medicated patch kit 1500 and four views of a medicated patch 1502 in accordance with one exemplary configuration. Shown is a trimetric view 1510, a top view 1512, and an exploded side view 1514 of the medicated patch kit 1500 and a trimetric view 1516, a top view 1518, and a front view 1520, and a back view 1522 of the medicated patch 1502. The medical patch assembly 1500 comprises the medicated patch 1502 housed within a mounting flange 1504. The medicated patch kit 1500 includes a void 1506 which may be configured to allow an internal catheter to fit into a passage 1508 of the medicated patch kit 1500. The medicated patch may comprise of a membrane made of a material such as a semi-porous membrane which may contain a topical chemical agent which can be utilized to alleviate pains and/or prevent infection in a patient. Topical chemical agents which may be used include: LMX 4 (4% lidocaine, Liposomal carrier), a generic form of EMLA (2.5% lidocaine and 2.5% prilocaine eutectic mixture), Topicain (4% lidocaine, amide, translucent microemulsion gel), LET (4% lidocaine, 1:2000 epinephrine, 0.5% tetracaine, gel, methylcellulose base), or BLT (20% benzocaine, 6% lidocaine, 4% tetracaine, emollient base).

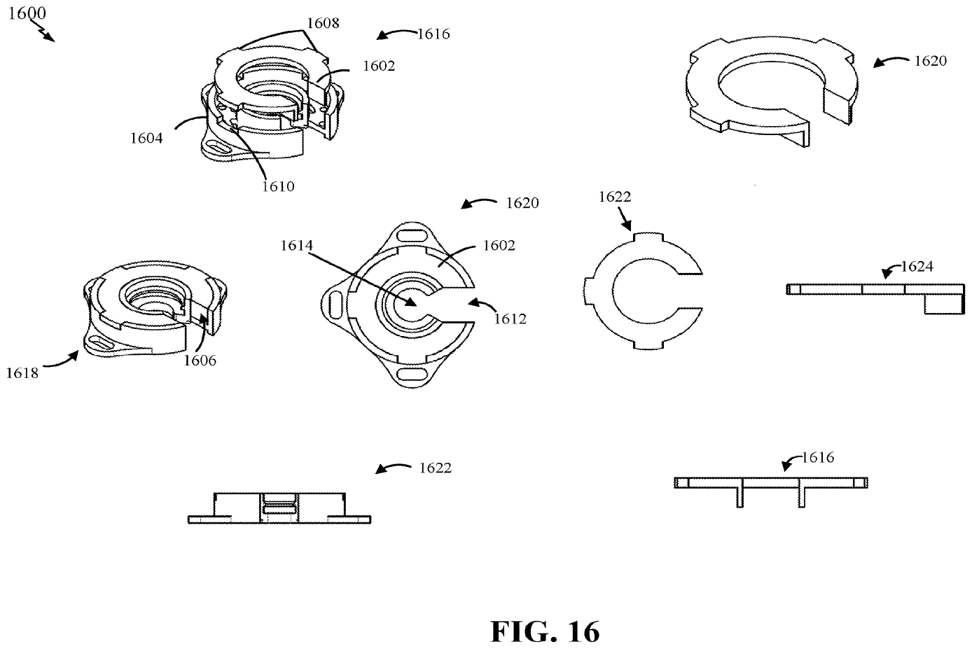

FIG. 16 illustrates four views of a medication cap kit 1600 and four views of the medical treatment pack 1602 in accordance with one exemplary configuration. Shown is an exploded trimetric view 1616, a trimetric view 1618, a top view 1620, and a side view 1622 of the medication cap kit 1900, and a trimetric view 1620, a top view 1622, and a side view 1624, and a front view 1616 of the medication patch 1602. The medication cap kit 1600 comprises the medication cap 1602 and a mounting flange 1604. The medication cap 1602 may be coupled to the mounting flange 1604 via a plurality of attachment tabs 1608 which interface with a plurality of notches 1610 on the mounting flange 1604. The medication cap kit 1600 may also include a void 1612 which may be configured to allow an internal catheter to fit into a passage 1614 of the medicated patch kit 1600. The mounting flange 1604 may include a trough 1606 which can serve as a medical treatment dispenser and can house a medical treatment. The medical treatment may comprise a topical chemical agent which can be utilized to alleviate pains and/or prevent infection in a patient. The medication cap 1602 may be utilized to contain the medical treatment placed within the trough 1606.

Therefore, as described above, a tissue interface is provided that can be coupled with an external catheter and an internal catheter to drain fluid from within a surgical site or cavity. These catheters may be made of a soft and flexible material, such as silicone elastomer. The material may also be impregnated with barium to produce a radiopacity enabling localization of either or both catheters on x-ray.

In some configurations, the tissue interface includes an attachment system, a drain coupling, and a mounting flange. The tissue interface and/or any of the attachment system may be made of a durable, low-porosity injection-molded plastic. The plastic may also be bioinert to prevent allergic reactions, such as PVC or polyethylene.

The mounting flange provides a secure conduit through which the internal catheter and external catheter coupled to the drain coupling and attachment system respectively, may pass. The catheters coupled to the connectors are fitted onto the mounting flange by passing the internal catheter through the void. Disassembly of attachment system from the mounting flange can be performed utilizing a snap fitting, a Luer lock, a screw and threaded hole system, or a similar method.

The present disclosure has described one or more configurations, and it should be appreciated that many equivalents, alternatives, variations, and modifications, aside from those expressly stated, are possible and within the scope of the invention.

It is to be understood that the disclosure is not limited in its application to the details of construction and the arrangement of components set forth in the accompanying description or illustrated in the accompanying drawings. The disclosure is capable of other configurations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular configurations or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or configurations. Further, references to particular rotational or other movements (e.g., counterclockwise rotation) is generally intended as a description only of movement relative a reference frame of a particular example of illustration.

In some configurations, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single-or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, configurations of the disclosure can be implemented as a set of instructions, tangibly embodied on a non-transitory computer-readable media, such that a processor device can implement the instructions based upon reading the instructions from the computer-readable media. Some configurations of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular configurations of the disclosure. Further, in some configurations, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as configurations of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

This discussion is presented to enable a person skilled in the art to make and use configurations of the disclosure. Various modifications to the illustrated examples will be readily apparent to those skilled in the art, and the generic principles herein can be applied to other examples and applications without departing from the principles disclosed herein. Thus, configurations of the disclosure are not intended to be limited to configurations shown, but are to be accorded the widest scope consistent with the principles and features disclosed herein and the claims below. The accompanying detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” Further, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of each of A, B, and C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C. In general, the term “or” as used herein only indicates exclusive alternatives (e.g. “one or the other but not both”) when preceded by terms of exclusivity, such as “either,” “one of,” “only one of,” or “exactly one of.”

Also as used herein, unless otherwise specified or limited, the terms “about” and “approximately,” as used herein with respect to a reference value, refer to variations from the reference value of ±15% or less (e.g., ±10%, ±5%, etc.), inclusive of the endpoints of the range. Similarly, the term “substantially equal” (and the like) as used herein with respect to a reference value refers to variations from the reference value of less than ±30% (e.g., ±20%, ±10%, ±5%) inclusive. Where specified, “substantially” can indicate in particular a variation in one numerical direction relative to a reference value. For example, “substantially less” than a reference value (and the like) indicates a value that is reduced from the reference value by 30% or more, and “substantially more” than a reference value (and the like) indicates a value that is increased from the reference value by 30% or more.

1. A tissue interface for connecting a drain to a patient at a skin surface, the tissue interface comprising:
a mounting flange comprising:
a passage extending through the mounting flange and configured to receive at least one catheter forming a drain tube therethrough to extend through an opening in the skin surface of the patient to drain fluid from within the patient;
a plurality of suture tabs arranged about the passage to secure the mounting flange against the skin surface of the patient; and
an attachment system configured to removably engage the at least one catheter to secure the at least one catheter to the mounting flange.

2. The tissue interface of claim 1, further comprising medical treatment system configured to deliver a medical treatment to the skin surface proximate to the opening in the skin surface.

3. The tissue interface of claim 2, wherein the medical treatment system includes a trough extending around the passage.

4. The tissue interface of claim 3, wherein the trough includes a plurality of perforations to allow the medical treatment to pass through the mounting flange to the skin surface.

5. The tissue interface of claim 4, wherein the perforations include a plurality of opening sizes.

6. The tissue interface of claim 5, wherein the opening sizes include a circular opening and an elongated opening, and wherein the elongated opening is arranged proximate to the passage to deliver an increased amount of medical treatment to the opening in the skin surface relative to portions of the skin surface arranged away from the opening in the skin surface.

7. The tissue interface of claim 1, wherein the plurality of suture tabs are arranged about a periphery of the mounting flange or on three sides of the mounting flange.

8. The tissue interface of claim 1, wherein the mounting flange includes a void configured to slide the at least one catheter into and out from the passage of the mounting flange.

9. The tissue interface of claim 1, further comprising at least one lock configured to engage the attachment system to removably connect the catheter to the mounting flange.

10. The tissue interface of claim 9, wherein the at least one lock includes a snap fitting to removably connect the attachment system to the mounting flange.

11. The tissue interface of claim 9, further comprising a Luer lock to removably connect the attachment system to the mounting flange.

12. The tissue interface of claim 9, wherein the mounting flange is semi-rigid to deform to engage the skin surface.

13. A kit for connecting a drain to a patient at a skin surface, the kit comprising:
a mounting flange comprising:
a passage extending through the mounting flange and configured to receive at least one catheter therethrough to extend through an opening in the skin surface of the patient to drain fluid from within the patient;
a plurality of suture tabs arranged about the passage to secure the mounting flange against the skin surface of the patient;
a medical treatment system configured to deliver a medical treatment arranged therein to the skin surface proximate to the opening in the skin surface; and
an attachment system configured to removably connect to the mounting flange and engage the at least one catheter to secure the at least one catheter to the mounting flange.

14. The kit of claim 13, further comprising medical treatment pack includes at least one of an antibiotic or an anesthetic.

15. The kit of claim 14 wherein the medical treatment system includes a trough formed in the mounting flange and wherein the medical treatment pack is formed in a ring configured to be arranged in the trough.

16. The kit of claim 15, wherein the ring includes a removable cover of a membrane through which the at least one of the antibiotic or the anesthetic is configured to pass to be delivered to the skin surface through perforations in the mounting flange.

17. The kit of claim 13, wherein the mounting flange includes a void configured to slide the at least one catheter into and out from the passage of the mounting flange.

18. The kit of claim 13, further comprising a lock configured to engage the catheter to removably connect to the mounting flange.

19. The kit of claim 18, wherein the lock includes at least one of a snap fitting to removably connect the at least one catheter to the mounting flange or a Luer lock to removably connect the at least one catheter to the mounting flange.

20. A method of installing a drain in a patient, the method comprising:
positioning at least one catheter to arrange a distal end to drain fluid from inside the patient;
positioning the at least one catheter to arranged a proximal end to extend externally from the patient to dispel the fluid drained from inside the patient from the distal end from the patient;
arranging the at least one catheter to extend through a passage of a mounting flange arranged to secure the at least one catheter to a tissue or skin interface of the patient;
suturing the mounting flange to the tissue or skin interface of the patient using a plurality of suture tabs extending from the mounting flange; and
attaching the at least one catheter to the mounting flange using an attachment system.
